(19) United States
(12) Patent Application Publication
(10) Pub. No.: US 2012/0202892 A1
(43) Pub. Date: Aug. 9, 2012

(54) Cannabinoid-Containing Compositions and Methods for Their Use

(75) Inventors: Audra L. Stinchcomb, Lexington, KY (US); Stan Lee Banks, Frankfort, KY (US)

(73) Assignee: University of Kentucky Research Foundation, Lexington, KY (US)

(21) Appl. No.: 13/451,666

(22) Filed: Apr. 20, 2012

Related U.S. Application Data
(63) Continuation of application No. 12/399,392, filed on Mar. 6, 2009, now abandoned.
(60) Provisional application No. 61/068,328, filed on Mar. 6, 2008.

(51) Int. Cl. A61K 31/05; A61P 19/02; A61P 29/00

(52) U.S. Cl. 514/733

(57) ABSTRACT

This invention relates to cannabinoid-containing compositions, particularly cannabinoid-containing gel formulations and methods for the treatment of traumatic injury, e.g., strains, sprains and contusions, and disease conditions, e.g., arthritis, particularly osteoarthritis. The methods involve topically applying a cannabinoid or a cannabinoid-containing composition to a subject's skin near, or distant from, the area of the injury or the area affected by the disease condition, e.g., an arthritic joint. The cannabinoid-containing composition is preferably a pharmaceutically acceptable gel containing a therapeutically effective amount of a cannabinoid sufficient to alleviate the symptoms associate with the injury or disease condition.

### CROSS-REFERENCE TO RELATED APPLICATIONS

This application is a continuation of U.S. application Ser. No. 12/399,392, filed Mar. 6, 2009, which claims priority under 35 U.S.C. §119 to provisional application No. 61/068,328, filed Mar. 6, 2008, which is incorporated herein in its entirety by reference.

### TECHNICAL FIELD

The present invention generally relates to the transdermal delivery of cannabinoids. More particularly, the present invention relates to methods and materials for transdermally delivering cannabinoids, particularly cannabidiol, to treat symptoms, e.g., inflammation, pain and discomfort, associated with or caused by traumatic injury or disease conditions, e.g., strains, sprains, contusions and arthritis, particularly osteoarthritis.

### BACKGROUND OF THE INVENTION

Arthritis is classified as one of the rheumatic diseases, which are related in that they have a tendency to affect the joints, muscles, ligaments, cartilage, and tendons, and many have the potential to affect other internal body areas. The cause of arthritis ranges from wear and tear and traumatic injury of cartilage (contributing to, e.g., osteoarthritis) to inflammation due to metabolic abnormalities (such as gout and pseudogout), hereditary, infections and overactive immune systems (associated with rheumatoid arthritis and systemic lupus erythematosus).

An estimated 46 million adults in the United States have self-reported and doctor diagnosed forms of arthritis and the problem is becoming an even bigger concern as the baby boomers age. Twenty eight million adults have the most common forms of arthritis: osteoarthritis and rheumatoid arthritis (Lawrence et al., (2008) Arthritis Rheum 58:26-35). According to the US Census as of December of 2001, the World population of people of 65 and older is increasing at a rate of 800,000/month. The United States is ranked 32nd with 13% of the current population over the age of 65. It is estimated that from the year 2000 to 2030 the number of people over 65 will be increased by 102%. Osteoarthritis is estimated to cost the US approximately 3.4-13.2 billion dollars a year including medical costs plus lost productivity (Leigh et al., (2001) J Rheumatol 28: 1647-1654.). This debilitating disease condition is often untreatable, leading to limited performance of daily tasks and lower quality of life. No matter which form of arthritis is involved, pain is a constant accompanying factor in every case. Arthritis and its joint pain can be manifested as hip pain, knee pain, hand pain, or wrist Pain, as well as joint pain elsewhere in the body.

The primary treatments for osteoarthritis pain are non-steroidal anti-inflammatory drugs (NSAIDs), administered either orally or topically, which have been associated with toxic side effects due to their role with inhibition of cyclo-oxgenase (COX) enzymes, particularly COX-2. Transdermally delivered opioids, such as fentanyl, provide pain relief for arthritis patients (Langford et al., (2006) Arthritis Rheum 54: 1829-1837). Intraarticular injections of glucocorticoid and hyaluronic acid (Gerwin et al., (2006) Adv Drug Deliv Rev 58: 226-242) are also available.

Cannabinoid receptors CB1 and CB2 are primarily found in the brain (nervous system) and immune system, respectively. Both CB receptors play a role in pain, however, CB1 receptor activation tends to be associated with psychoactive side effects whereas CB2 receptor activation is not (Clayton et al., (2002) Pain 96: 253-260). CB2 activation produced analgesia and reduced inflammation in rats (Clayton et al., 2002). Cannabinoids like Δ9-tetrahydrocannabinol (THC) work at either CB1 or CB2 as agonists. Some cannabinoids, such as cannabidiol (CBD), have little binding affinity for CB1 and CB2 receptors leaving their role in receptor activation or deactivation not completely understood. Others have suggested that CBD actions are actually mediated via a transient receptor potential vanilloid type 1 (TRPV1) (Costa et al., (2004b) Br J Pharmacol 143: 247-250).

When THC has been administered orally, side effects and lack of positive response lead to patients discontinuing in the trial (Attal et al. (2003) Eur J Pain 8: 173-177; Marian Jr et al. (2003) Curr Opin Pharmacol 3: 62-67). The lack of positive response is often due to peak plasma level-related adverse effects. Side effects occur when effective/higher doses of the THC were administered to the point patients could no longer function in daily tasks (Attal et al. (2003) Eur J Pain 8: 173-177 ; Marian Jr et al. (2003) Curr Opin Pharmacol 3: 62-67. CBD, while structurally similar to THC (FIG. 1), is a non-psychoactive component of marijuana. CBD has been examined for its therapeutic potential for neuropathic pain, cancer pain, multiple sclerosis, and inflammation (Burstein and Zurier (2009) AAPS J. Epub ahead of press. PMID: 19199042). CBD is more potent than aspirin (360×) and THC (590×).

Canabidiol (“CBD”) is the major nonpsychoactive component of cannabis. Malfait et al. (2000) (Proc. Natl. Acad. Sci. 97(17):9561-9566) explored the therapeutic potential of intraperitoneally (ip) and orally administered CBD in a murine model for rheumatoid arthritis, wherein mice were administered type II collagen in complete freund's adjuvant (CIA) (Courtenay et al. (1980) Nature (London) 283:666-668). Malfait et al. report that CBD had a beneficial therapeutic action on established CIA when administered systemically. Rats, which had received a complete Freund's adjuvant (CFA) intraplantar injection, were administered 20 mg/kg of CBD orally for 7 consecutive days (Costa et al. (2007) Eur J Pharmacol 556: 75-83). Results showed a 50% decrease in thermal and mechanical withdrawal latency and that repeated dosing was needed to decrease pain.

### SUMMARY OF THE INVENTION

Cannabinoids have been proven effective in relief of inflammatory pain. Topical application and transdennal delivery of cannabinoids, particularly CBD, allows for a more controlled drug delivery rate. Delivery of pain controlling drugs through the skin allows for targeting specific sites and joints for treatment. An increase in the duration of the effect of such drugs would significantly improve clinical efficacy. The results presented herein demonstrate of the in vivo effects of transdermal delivery of a cannabinoid-containing composition to CFA mono-arthritic knee joints in Sprague-Dawley rats. Our results demonstrate that transdermal delivery of the cannabinoid-containing composition, particularly a CBD-containing gel, decreased inflammation and pain without inducing abnormal behavior or other adverse effects.

The present invention relates to methods for alleviating the symptoms associated with arthritis, particularly osteoarthritis. The methods involve providing a cannabinoid-containing composition suitable for topical application and delivering the cannabinoid to a subject in need thereof. The cannabinoid may be one selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2 and Δ⁹-THC. Preferably the cannabinoid is cannabidiol and the cannabinoid containing composition is a pharmaceutically acceptable gel.

### BRIEF DESCRIPTION OF THE DRAWINGS

FIG. 1 depicts the structure of cannabidiol.

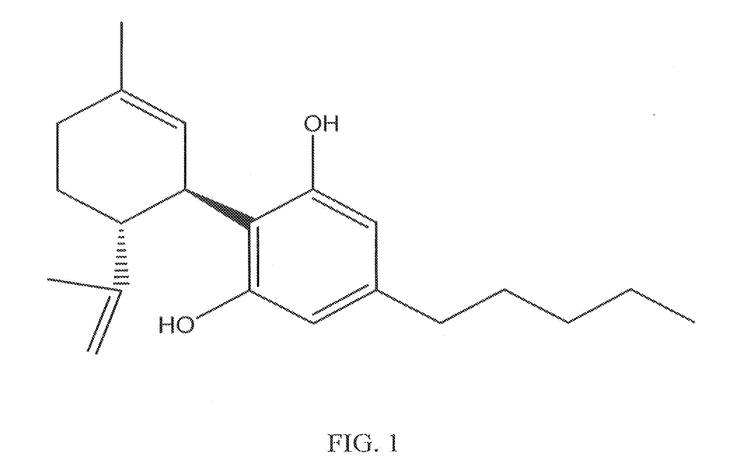

FIG. 2A-B compares the paw withdrawal latency from (A) CFA induced rats treated with CBD and (B) normal rats treated with CBD. Data are represented as mean±SD. In FIG. 1A, a significant improvement (p<0.05) was observed in rats receiving 6.2 mg/d (9.2±0.7) and 62.3 mg/d (9.1±0.2) treatments as compared to rats receiving the vehicle gel (7.1±0.6), 0.6 mg/d (7.8 ±0.5), and 3.1 mg/d (7.6±0.8) treatments (normal+vehicle [], n=4; 0.6 mg [x], n=5; 3.1 mg [▴], n=5; 6.2 mg [▪], n=5; 62.3 mg [♦], n=3, CFA+vehicle [ [symbol] , n=11). In FIG. 1B, the PWL in normal animals was not affected by any CBD dose (normal+vehicle [], n=4; 0.6 mg [x], n=5; 3.1 mg [▴], n=5; 6.2 mg [▪], n=5).

FIG. 3A-F compares the exploratory activities in normal control rats treated with different doses of CBD: (A) rearing events, (B) rearing time, (C) total beams broken, (D) distance traveled, (E) active time, (F) resting time [light gray bar=pre-drug, black bar=post-drug]. Data are represented as mean±SD. (0 mg, n=4; 0.6 mg, n=6; 3.1 mg, n=15; 6.2 mg, n=3; 62.3 mg, n=1). No significant differences (p>0.05) were determined among the different dose treatment groups for the six parameters.

### DETAILED DESCRIPTION OF THE INVENTION

The methods of the present invention involve applying a cannabinoid or cannabinoid containing composition to the skin of a subject in need thereof to transdermally deliver a therapeutically effective amount of cannabinoid sufficient to alleviate the symptoms of osteoarthritis. The cannabinoid composition includes at least one cannabinoid selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2 and Δ⁹-THC.

The present invention overcomes the problems associated with existing drug delivery systems by delivering cannabinoids transdermally. Preferably, the cannabinoids are delivered transdermally via a pharmaceutically acceptable cannabinoid-containing composition, e.g., a cannabinoid-containing gel, to reduce harmful side effects and avoid gastrointestinal (first-pass) metabolism of the drug by the patient.

A first aspect of the invention provides a method for relieving symptoms associated with osteoarthritis in a subject, particularly a mammalian subject, wherein the symptoms of osteoarthritis include at least one of inflammation, pain or discomfort. The method comprises the steps of selecting at least one cannabinoid from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2 and Δ⁹-THC, and applying the cannabinoid to the skin of a subject in need thereof in an amount and for a time sufficient to alleviate the symptoms of osteoarthritis. The cannabinoid containing composition may contain 0.5-25% w/w, 1%-20% w/w, 1%-15% w/w, 1%-10% w/w, 5%-15%, 5%-10% or 7.5%-12.5% w/w cannabinoid, preferably 1%-10% w/w cannabinoid. Desired systemic levels of cannabinoid may be achieved by applying the cannabinoid-containing composition to a subject's skin for a time and in an amount sufficient to achieve a desired systemic level of cannabinoid, e.g., a systemic level of about 5-50 mg/day, about 5 mg-40 mg/day, about 10 mg-40 mg/day, or about 36 mg/day in the subject.

The cannabinoid may also be combined with a material(s) that enhances the cannabinoid's permeation of the skin, a permeation enhancer, and the combination applied to the subject's skin. Illustrative permeation enhancers that can be used with the cannabinoid include isopropyl myristate, propylene glycol monolaurate, diethylene glycol monoethyl ether, an oleoyl macrogolglyceride, a caprylocaproyl macrogolglyceride, an oleyl alcohol, ethanol and oleic acid.

The cannabinoid with or without the permeation enhancer, may also be combined with a pharmaceutically acceptable gel forming material to form a cannabinoid containing gel formulation, and the formulation is applied topically to the subject's skin for transdermal delivery of a therapeutically effective amount of the cannabinoid to the subject in need. Many commercial polymers are available that are suitable for forming pharmaceutically acceptable gels. The gel forming material may be e.g., anionic polymers such as polyacrylic acid (CARBOPOL™ by Noveon, Inc., Cleveland, Ohio), carboxypolymethylene, carboxymethylcellulose and the like, including derivatives of Carbopol™ polymers, such as Carbopol™ Ultrez 10, Carbopol™ 940, Carbopol™ 941, Carbopol™ 954, Carbopol™ 980, Carbopol™ 981, Carbopol™ ETD 2001, Carbopol™ EZ-2 and Carbopol™ EZ-3, and other polymers such as Pemulen™ polymeric emulsifiers, and Noveon™ polycarbophils. Additional gel forming materials, enhancers and adjuvants may generally be found in Remington's The Science and Practice of Pharmacy as well as the Handbook f Pharmaceutical Excipients, Arthur H. Kibbe ed. 2000. Gel forming materials or gelling agents are present in an amount sufficient to provide the desired theological properties of the composition. Illustratively, one or more pharmaceutically acceptable gel forming materials or gelling agents are present in a total amount by weight of about 0.1%, about 0.25%, about 0.5%, about 0.75%, about 0.9%, about 1%, about 1.25%, about 1.5%, about 1.75%, about 2.0%, about 2.25%, about 2.5%, about 2.75%, about 3.0%, about 3.25%, about 3.5%, about 3.75%, about 4.0%, about 4.25%, about 4.5%, about 4.75%, about 5.0%, about 5.25%, about 5.5%, about 5.75%, about 6.0%, about 6.25%, about 6.5%, about 6.75%, about 7.0%, about 7.25%, about 7.5%, about 7.75%, about 8.0%, about 8.25%, about 8.5%, about 8.75%, about 9.0%, about 9.25%, about 9.5%, about 9.75%, about 10%, about 11%, about 11.5%, about 12%, about 12.5%, about 13%, about 13.5%, about 14%, about 14.5% or about 15%.

In one embodiment a neutralizing agent is optionally present to assist in forming a gel. Suitable neutralizing agents include sodium hydroxide (e.g., as an aqueous mixture), potassium hydroxide (e.g., as an aqueous mixture), ammoniuin hydroxide (e.g., as an aqueous mixture), triethanolamine, tromethamine (2-amino2-hydroxymethyl-1,3propanediol), aminomethyl propanol (AMP), tetrahydroxypropyl ethylene diamine, diisopropanolamine, Ethomeen C-25 (Armac Industrial Division), Di-2 (ethylhexyl)amine (BASF-Wyandotte Corp., Intermediate Chemicals Division), triamylamine, Jeffamine D-1000 (Jefferson Chemical Co.), b-Dimethylaminopropionitrite (American Cyanamid Co.), A.rmeen CD (Armac Industrial Division), Alamine 7D (Henkel Corporation), dodecylamine and morpholine. The neutralizing agent is present in an amount sufficient to form a gel which is suitable for contact with the skin of a mammal.

The cannabinoid may also be applied with another pain relieving analgesic composition, e.g., an opiate, NSAID or COX-2 specific inhibitor, wherein the concentration of the other analgesic composition is sufficient to further alleviate the symptoms associated with osteoarthritis. The analgesic may be combined with the cannabinoid and the gel and applied to the skin of a subject in need thereof.

The cannabinoid or cannabinoid-containing composition is applied to the subject's skin in an amount and for a time sufficient to alleviate the symptoms, e.g., inflammation, pain and/or discomfort, that are associated with an injury e.g. a strain, sprain, contusion, or or disease condition, e.g., arthritis, particularly osteoarthritis. The cannabinoid or cannabinoid-containing composition may be applied in a single dose or multiple doses for one or more days. The cannabinoid-containing composition may be applied for a time and in an amount to achieve systemic levels of cannabinoid in the subject, e.g., systemic levels of about 5-50 mg/day, about 5 mg-40 mg/day, about 10 mg-40 mg or about 36 mg/day in the subject. Preferably the cannabinoid-containing composition is applied once per day to alleviate the subject's symptoms.

Although the cannabinoid-containing composition may be applied to skin in the area affected by the injury or disease condition, e.g., an osteoarthritic joint, it may also be applied to other areas of the body distant or not immediately proximate to the area affected by the injury or disease condition, e.g., the subject's back, abdomen, chest, upper arms, thighs etc., and still deliver the desired amounts of cannabinoid to the subject. The cannabinoid-containing composition may be applied to the subject's skin until it is completely or almost completely, absorbed into the skin.

The subject may be a mammalian subject, e.g., a rodent, a guinea pig, a cat, a dog, a horse, a cow, a pig, or a primate e.g., a chimpanzee, an ape or a human having osteoarthritis or having pain or discomfort associated with osteoarthritis.

A second aspect of the invention provides a viscous flowable pharmaceutically acceptable gel comprising a gel forming material and the cannabinoid. The gel may also comprise a permeation enhancer. The cannabinoid with or without the permeation enhancer may be suspended within the gel or may be dissolved within the gel. The cannabinoid may be selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2 and Δ⁹-THC. The permeation enhancer may be e.g., isopropyl myristate, propylene glycol monolaurate, diethylene glycol monoethyl ether, an oleoyl macrogolglyceride, caprylocaproyl macrogolglyceride, and an oleyl alcohol, ethanol and oleic acid. The pharmaceutically acceptable gel material may be selected from the group consisting of carbomers, e.g., Carbopol, Pemulen, and Noveon polymers. The gel may also be combined with one or more analgesics. The analgesic may be e.g. an opiate, a NSAID, or a COX-2 specific inhibitor. The concentration of the analgesic within the gel is sufficient to further alleviate the symptoms associated with osteoarthritis.

A third aspect of the invention provides a method for increasing the concentration of cannabinoids or cannabinoid metabolites in a subject, comprising contacting the subject's skin with a cannabinoid selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2 and Δ⁹-THC. The method may also comprise the step of contacting the subject's skin with the cannabinoid and a permeation enhancer e.g., isopropyl myristate, propylene glycol monolaurate, diethylene glycol monoethyl ether, an oleoyl macrogolglyceride, a caprylocaproyl macrogolglyceride, and an oleyl alcohol, ethanol and oleic acid. The cannabinoid with or without the permeation enhancer are preferably comprised in a pharmaceutically acceptable gel. The gel may be applied to the subject's skin until it is completely or almost completely, absorbed into the skin.

“Alleviate” as used herein, is meant to include complete elimination as well as any clinically or quantitatively measurable reduction in the subject's symptoms and/or discomfort.

“Cannabinoid,” as used herein, is meant to include compounds which interact with the cannabinoid receptor and various cannabinoid mimetics, such as certain tetrahydropyran analogs (e.g., Δ⁹-tetrahydrocannabinol, 66 ⁸-tetrahydrocannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo [b,d]pyran-9-one, (−)-(3S,4S)-7-hydroxy-Δ⁶-tetrahydrocannabinol-1,1-dimethylhept-yl, (+)-(3S,4S)-7-hydroxy-Δ⁶-tetrahydrocannabinol-1,1-dimethylh-eptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro-6-methy-1-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenylmethanone), certain open pyran ring analogs (e.g., 2-[3-methyl-6-(1-methylethenyl)-2-cyclohexen-1-yl]-5-pentyl-1,3-benzenedi-ol and 4-(1,1-dimethylheptyl)-2,3′-dihydroxy-6′alpha-(3-hydroxypropyl)-1′,-2′,3′,4′,5′,6′-hexahydrobiphen-yl), as well as their pharmaceutically acceptable salts, solvates, metabolites (e.g., cutaneous metabolites), and metabolic precursors. Further examples of “cannabinoids” include those compounds described in the references cited below.

“Δ⁹-THC,” as used herein, is meant to refer to Δ⁹-tetrahydrocannabinol as well as to its pharmaceutically acceptable salts, solvates, metabolites (e.g., cutaneous metabolites), and metabolic precursors. Δ⁹-tetrahydrocannabinol is marketed under the generic name “dronabinol.”

“Cannabinol,” as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6H-dib-enzo[b,d]pyran-1-ol. The synthesis of 6,6,9-trimethyl-3-pentyl-6H-dibenzo[-b,d]pyran-1-ol is described in, for example, Novak at al., Tetrahedron Letters, 23:253 (1982), which is hereby incorporated by reference.

“Cannabidiol,” as used herein, is meant to refer to 2-[3-methyl-6-(1-methylethenyl)-2-cyclohexen-1-yl]-5-pentyl-1,3-benzenedi-ol as well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenedi-ol. The synthesis of 2-[3-methyl-6-(1-methylethenyl)-2-cyclohexen-1-yl]-5-pentyl-1,3-benzenediol is described, for example, in Petilka et al., Helv. Chim. Acta, 52:1102 (1969) and in Mechoulam et al., J. Am. Chem. Soc., 87:3273 (1965), which are hereby incorporated by reference.

“Nabilone,” as used herein, is meant to refer to 3-(1,1-dimethylheptyl)-6,6a,7,8,10,10a-hexahydro-1-hydroxy-6,6-dimethyl-9-H-dibenzo[b,d]pyran-9-one as well as to pharmaceutically acceptable salts, solvates, metabolites (e.g., cutaneous metabolites), and metabolic precursors of 3-(1,1-dimethylheptyl)-6,6a,7,8, 10,10a-hexahydro-1-hydroxy-6,6-dimethyl-9H-dibenzo[b,d]pyran-9-one. 3-(1,1-dimethylheptyl)-6,6a,7,8,-10,10a-hexahydro-1-hydroxy-6,6-dimethyl-9H-dibenzo[b,d]pyran-9-one is approved for use in the United Kingdom for treating nausea and vomiting associated with chemotherapy, and its preparation is described, for example, in U.S. Pat. No. 3,968,125 to Archer, which is hereby incorporated by reference.

“Levonantradol,” as used herein, is meant to refer to (−)-(6S,6aR,9R,10aR)-5,6,6a,7,8,9,10,10a-octahydro-6-methyl-3-[(R)-1-meth-yl-4-phenylbutoxy]-1,9-phenan-thridinediol 1-acetate, as well as to pharmaceutically acceptable salts, solvates, metabolites (e.g., cutaneous metabolites), and metabolic precursors of (−)-(6S,6aR,9R, OaR)-5,6,6a,7,8,9,10,10a-octahydro-6-methyl-3-[(R)-1-methyl-4-phenylbuto-xy]-1,9-phenanthridinediol 1-acetate. (−)-(6S,6aR,9R,10aR)-5,6,6a,7,8,9,10,10a-octahydro-6-methyl-3-[(R)-1-methyl-4-phenylbuto-xy]-1,9-phenanthridinediol 1-acetate is particularly useful in pain control, and its synthesis is described in Belgian Pat. No. 854,655, which is hereby incorporated by reference; in U.S. Pat. Nos. 4,206,225, 4,232,018, and 4,260,764, each to Johnson, which are hereby incorporated by reference; in U.S. Pat. No. 4,235,913 to Johnson et al., which is hereby incorporated by reference; in U.S. Pat. No. 4,243,674 to Bindra, which is hereby incorporated by reference; and in U.S. Pat. Nos. 4,263,438, 4,270,005, and 4,283,569, each to Althuis et al., which are hereby incorporated by reference.

“(−)-HU-210,” as used herein, is meant to refer to (−)-(3S,4S)-7-hydroxy-Δ⁶-tetrahydrocannabinol-1,1-dimethylhept-yl as well as to pharmaceutically acceptable salts, solvates, metabolites (e.g., cutaneous metabolites), and metabolic precursors of (−)-(3S,4S)-7-hydroxy-Δ⁶-tetrahydrocannabinol-1,1-dimethylhept-yl. (−)-(3S,4S)-7-hydroxy-Δ⁶-tetrahydro-cannabinol-1,1-dimethylh-eptyl is particularly useful in pain control, and its preparation is described in U.S. Pat. Nos. 4,876,276 and 5,521,215, each to Mechoulam et al., which are hereby incorporated by reference.

“(+)-HU-210,” as used herein, is meant to refer to (+)-(3S,4S)-7-hydroxy-Δ⁶-tetrahydrocannabinol-1,1-dimethylhept-yl as well as to pharmaceutically acceptable salts, solvates, metabolites (e.g., cutaneous metabolites), and metabolic precursors of (+)-(3S,4S)-7-hydroxy-Δ⁶-tetrahydrocannabinol-1,1-dimethylhept-yl. (+)-(3S,4S)-7-hydroxy-Δ⁹-tetra-hydrocannabinol-1,1-dimethylh-eptyl is sometimes referred to as HU-211 and/or dexanahinol; it is an antagonist of the N-methyl-D-aspartate receptor; and its preparation is described in U.S. Pat. Nos. 4,876,276 and 5,521,215, each to Mechoulam et al., which are hereby incorporated by reference.

“11-hydroxy-Δ⁹-THC,” as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahydrocannabinol is a more hydrophilic, psychoactive metabolite of Δ⁹-tetrahydrocannabinol, and its laboratory synthesis has been described in Siegel et al., J. Org. Chem., 54:5428 (1989), which is hereby incorporated by reference.

“Δ⁸-THC-11-oic acid,” as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabino-1-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa) and is produced from 6a,7,10,10a-tetrahydro-6,6,9-trimethyl-3-pentyl-6H-dibenzo[b,d]pyran-1-o-1 via a series of biotransformations mediated primarily by mammalian liver enzymes. A8-tetrahydrocannabinol-11-oic acid can also be produced synthetically by reference to the synthetic schemes set forth in U.S. Pat. No. 6,162,829 to Burstein, which is hereby incorporated by reference. Δ⁸-tetrahydrocannabin-ol-11-oic acid is more hydrophilic than 6a,7,10,10a-tetrahydro-6,6,9-trimethyl-3-pentyl-6H-diben-zo[b,d]pyran-1-ol, and it has analgesic activity.

“CP 55,940,” as used herein, refers to 4-(1,1-dimethylheptyl)-2,3′-dihydroxy-6′alpha-(3-hydroxypropyl)-1′,2′,3′,4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′alpha-(3-hydroxypropyl)-1′,2′,3′,4′,5′,6′-hexahydro-biphenyl is sometimes referred to as (−)-cis-3-[2-Hydroxy-4-(1,1-dimethylheptyl)phenyl]-trans-4-(3-hydroxyprop-yl)cyclohexanol, and it is commercially available from Tocris Cookson, Inc., Ellisville, Mo. Its preparation has been described in U.S. Pat. No. 4,371,720 to Johnson et al. and U.S. Pat. No. 4,663,474 to Urban, which arc hereby incorporated by reference.

“R(+)-WIN 55,212-2,” as used herein, refers to (R)-(+)-[2,3-dihydro-5-methyl-3-(4-morpholinylmethyl)-pyrrolo[1,2,3-de]-1-,4-benzoxazin-6-yl]-1-naphthalenyl-methanone, as well as to its pharmaceutically acceptable salts, solvates, metabolites (e.g., cutaneous metabolites), and metabolic precursors. (R)-(+)-[2,3-dihydro-5-methyl-3-(-4-morpholinylmethyl)-pyrrolo[1,2,3-de]-1,4-benzoxazin-6-yl]-1-naphthalenyl-methanone (in its mesylate form) is commercially available, for example, from Tocris Cookson, Inc., Ellisville, Mo., and from Research Biochemicals International, Natick, Mass.

The cannabinoid-containing composition may further include one or more additional cannabinoids. The one or more additional cannabinoids can be selected from the aforementioned list of cannabinoids or it (they) can be selected from cannabinoids which are not contained in the aforementioned list, such as Δ⁸-THC, high affinity cannabinoid receptor agonists (other than R(+)-WIN 55,212-2 and CP 55,940), and the like. Illustratively, the cannabinoid composition can include two or more cannabinoids, each being selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 to Martin et al., which is hereby incorporated by reference.

“Metabolites” of cannabinoids, as used herein, are meant to include compounds which are produced by the metabolic processes (e.g., cutaneous metabolic processes and/or systemic metabolic processes) of the subject's body. Suitable metabolites can be identified, for example, by studying the kinetics of drug enzymatic metabolism in skin homogenates. Illustratively, skin homogenates can be prepared from 250-μm dermatomed fresh healthy abdominal plastic surgery samples. The skin is homogenized (e.g., using a Polytron tissue homogenizer and ground glass homogenizer fitted with a glass pestle) in 4-(2-hydroxyethyl)-1-piperazineethanesulfonic acid (“HEPES”)-buffered Hanks' balanced salt solution. Whole homogenates can be used for these studies or, if significant mitochondrial or nuclear metabolism is found not to occur (e.g., by comparing the degree of metabolism in the supernatant the degree of metabolism in the whole homogenate), the studies can be carried out on only the supernatant fraction. The drug (solubilized in, for example, buffer, ethanol, dimethylsulfoxide, or combinations thereof) is then incubated with the homogenate (or supernatant) along with NADPH (or a generating system), NADH, MgCl₂, and bovine serum albumin. The total volume of ethanol in the reaction mixture should be small (e.g., under 2%) to help minimize ethanol's detrimental effects on the enzymes. After incubating for a period of time, the reaction is terminated with 15% trichloroacetic acid, and the drug and its metabolites are obtained by solid-phase extraction. The metabolite or metabolites formed can then be identified and assayed by any suitable method (e.g., HPLC).

The term “therapeutically effective amount” or “therapeutically and/or prophylactically effective amount” as used herein refers to an amount of a cannabinoid that is sufficient to elicit the required or desired therapeutic and/or prophylactic response. Preferably the “therapeutically effective amount” or “therapeutically and/or prophylactically effective amount” of cannabinoid is sufficient to alleviate the symptoms associated with osteoarthritis.

It is understood that a therapeutically and/or prophylactically effective amount of a drug for a subject is dependent inter alia on the body weight of the subject as well as other factors known to a person of ordinary skill in the art. A “subject” herein to which a therapeutic agent or composition thereof can be administered includes mammals such as a human subject of either sex and of any age, and also includes any nonhuman animal, particularly a domestic, farm or companion animal, illustratively a mouse, a cat, cow, pig, dog or a horse as well as laboratory animals such as guinea pigs and primates.

As used herein, the term “gel” or “gel matrix” means a type of reservoir or vehicle for the cannabinoid. A gel takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles), or a network of polymer chains. In some embodiments, a gel matrix may include hydrogels, organogels, and the like.

A “hydrogel” or “hydrogel matrix” refers to a three dimensional network of, for example, cross-linked hydrophilic polymers in the form of a gel and substantially composed of water.

Protocols for forming gels, including hydrogels, are well known in the art, as are protocols for forming gels and hydrogels comprising therapeutically effective amounts of one or more active agents.

A preferred gel formulation comprises about 1% w/w cannabinoid; about 70% w/w ethanol, about 20% w/w water, about 0.5% w/w isopropyl myristate, about 1% w/w Carbopol 980 and about 5% w/w 0.1N NaOH solution. The gel formulation may also comprise about 10% w/w cannabinoid; about 70% w/w ethanol, about 10% w/w water, about 0.5 w/w isopropyl myristate, about 1% w/w Carbopol 980 and about 5% w/w 0.1N NaOH solution.

As one skilled in the art will recognize, optimization of the method of the present invention will involve consideration of a variety of factors in selecting the cannabinoid to be used. One such factor is skin permeability. Several physicochemical factors influence the ability of cannabinoids to penetrate the skin. These include the cannabinoid's molecular weight, its molecular volume, its lipophilicity, its hydrogen bonding potentials, its polarity, etc.

As indicated above, the cannabinoid is delivered transdermally to the subject, for example, by topically applying the cannabinoid or cannabinoid-containing composition to the subject's skin. Generally transdermal delivery involves contacting the cannabinoid or cannabinoid-containing composition with the subject's skin under conditions effective for at least one of the provided cannabinoids to penetrate the skin.

The cannabinoid-containing composition can be formulated as a topical gel formulation. The topical gel formulations can include inert diluents and carriers as well as other conventional excipients, such as wetting agents, preservatives, and suspending and dispersing agents. In addition to the above, generally non-active components, topical gel formulations containing the cannabinoid can further include other active materials, particularly, active materials which have been identified as useful in the treatment of pain, discomfort, or other conditions associated with a subject's illness and which can usefully be delivered transdermally to the subject. For instance, such other active materials can include analgesics, such as opiates and other analgesic active materials which operate on non-cannabinoid receptors. Where, for example, opiates are included, transdermally deliverable opiates are particularly preferred. One example of a transdermally deliverable opiate is fentanyl. The topical formulation can be applied directly to the skin until it is completely or almost completely absorbed into the skin. The gel may be optionally covered (e.g., with a bandage of gauze) to minimize the likelihood of its being disturbed. Alternatively, the topical gel formulation can be coated on the surface of a bandage, gauze, etc., and the bandage, gauze, etc. can then be applied to the skin of the subject such that the topical gel formulation is in direct contact with the subject's skin. Preferably, the topical gel is not coated onto the surface of a bandage or gauze, etc. for application to the subjects skin.

The cannabinoid composition can also include one or more inhibitors of cannabinoid metabolism, particularly in cases where inhibition of cutaneous metabolism is needed to increase therapeutic drug levels. Such inhibitors of cannabinoid metabolism can include inhibitors of the P450 enzynies or other identified critical enzymatic processes. Suitable inhibitors of cannabinoid metabolism include, for example, essential oils which inhibit the activity of cytochrome P450 3 A in the skin, such as those described in U.S. Pat. No. 5,716,928 to Benet et al., which is hereby incorporated by reference. Some of these essential oils may also act as transdermal penetration enhancers, thus providing a dual mechanism of percutaneous penetration increase.

The present invention is further illustrated with the following examples.

### EXAMPLES

### A. Materials and Methods

### 1. Materials

CBD was a generous gift obtained from National Institute on Drug Abuse (“NIDA”). CFA was obtained from DIFCO Laboratories (Detroit, Mich.). Isopropyl myristate (IPM), sodium hydroxide, ethyl acetate (HPLC grade), and ammonium acetate (HPLC grade) were purchased through Fisher Scientific (Fairlawn, N.J.). Acetonitrile (ACN) (HPLC grade) was purchased from VWR (West Chester, Pa.). Absolute ethanol (USP grade) was purchased from Sigma-Aldrich (St. Louis, Mo.). Pre-purified nitrogen was purchased from Scott-Gross Company Inc (Lexington, Ky.). Carbopol® 980 was obtained from Noveon, Inc. (Cleveland, Ohio). Nanopure water was obtained from a Barnstead NANOpure® DIamond™ ultrapure water filtration system (Dubuque, Iowa).

### 2. Gel Preparation

Gels with and without 1% w/w or 10% w/w CBD were prepared in a similar manner. The respective amount of CBD was weighed and dissolved in 72.5% w/w ethanol. Once dissolved, 20.5% w/w nanopure water was added to the solution to and then isopropyl myristate (IPM) 0.5% w/w was added. Carbopol™ 980 0.9% w/w was added to the solution and then sonicated for 10 min to ensure complete incorporation of the Carbopol™ 980. Sodium hydroxide (0.1 N solution) 4.7% w/w was added to the sonicated solution to initiate thickening of the gel. The formulation was then sonicated for an additional 10 min, loaded into 1 mL syringes and sealed. The gels were made prior to the initial dosing and the pre-filled syringres were stored at ambient temperature in the dark for the entire week of application. The stability of the CBD containing gel at room temperature over 8 days was evaluated. No degradation was observed in the concentration of CBD in the gel. The CBD-containing gel failed to display any visible changes over the course of 8 days. The gel with and without CBD remained clear and colorless.

### 3. Animals

All animal procedures were approved by the University of Kentucky IACUC committee.

Experiments were performed using Sprague-Dawley male rats (260-280 g) purchased from Harlan Sprague Dawley, Inc. (Indianapolis, Ind.). Rats were housed in individual cages and allowed access to food and water ad libitum except during testing and dosing in a quiet room with a 12 h/12 h dark/light cycle.

### 4. Induction of Arthritis

To induce monoarthritis, rats were briefly anesthetized by isoflurane inhalation; the left knee joint cavity was injected with 0.1 mL of 2 mg/mL suspension of Mycobacterium tuberculosis (CFA) in 1:1 peanut oil and saline. Rats were monitored daily and knee joint inflammation and pain were assessed before CFA injection and daily beginning on day 3 after CFA (days 3-7).

### B. Assessment of Knee Joint Inflammation and Pain-Related Posture

### 1. Measurement of the Knee Joint Circumference

The circumference of the affected knee joints were measured in cm with a flexible tape wrapped around the center of the joint while the hind limbs were held in extension both before injection of CFA and after 4 and 7 days following the injection of CFA. The knee joint circumferences of rats treated with the CBD containing gel and the gel without CBD (the “vehicle gel”)were measured and compared (see Table 1).

The results presented in Table 1 demonstrate that the knee joint circumferences (cm) due to CFA induced inflammation were significantly decreased for rats receiving the 6.2 mg/d (6.6±0.2) and 62.3 mg/d (6.6±0.1) CBD doses (p<0.05) compared to those of the rats given vehicle gel respectively (7.2±0.3 and 7.0±0.0) (Table 1). Normal knee joint circumferences measured for each group prior to receiving vehicle gel or CBD were 5.8±0.2 and 5.7±0.2. There were no significant differences (p>0.05) in knee joint temperature among the treatment groups (Table 2).

TABLE 1
Assessment of CFA-induced knee joint inflammation after the application
of CBD or vehicle gel for four consecutive days (day 4)
Knee joint circumference (cm) [mean ± SD]

| | | CFA + PLA | CFA + CBD |
|---|---|---|---|
| Baseline | | 5.8 ± 0.2^{b} | 5.7 ± 0.2^{c} |
| 0.6 mg/d | CFA | 7.6 ± 0.2^{a} | 7.5 ± 0.3^{b} |
| | Post-CBD | 7.1 ± 0.2^{a} | 7.0 ± 0.3^{b} |
| 3.1 mg/d | CFA | 7.8 ± 0.2^{a} | 7.4 ± 0.4^{b} |
| | Post-CBD | 7.2 ± 0.4^{a} | 7.1 ± 0.3^{b} |
| 6.2 mg/d′ | CFA | 7.4 ± 0.4^{a} | 7.2 ± 0.3^{b} |
| | Post-CBD | 7.2 ± 0.3^{a} | 6.6 ± 0.2^{b}* |
| 62.3 mg/d | CFA | 7.1 ± 0.1^{a} | 7.2 ± 0.0^{a} |
| | Post-CBD | 7.0 ± 0.0^{a} | 6.6 ± 0.1^{a}* |

*p < 0.05
^{a}n = 3;
^{b}n = 5;
^{c}n = 8

TABLE 2
Assessment of CFA-induced knee joint temperature after the application
of CBD or vehicle gel for four consecutive days (day 4)
Knee joint temperature (° C.) [mean ± SD]

| | | CFA + PLA | CFA + CBD |
|---|---|---|---|
| Baseline | | 33.6 ± 1.1^{a} | 33.4 ± 0.2^{a} |
| 0.6 mg/d | CFA | 32.8 ± 0.4^{a} | 31.7 ± 1.7^{b} |
| | Post-CBD | 31.4 ± 0.2^{a} | 31.2 ± 0.7^{b} |
| 3.1 mg/d | CFA | 31.9 ± 0.8^{a} | 30.8 ± 0.9^{b} |
| | Post-CBD | 30.9 ± 1.4^{a} | 30.6 ± 0.6^{b} |
| 6.2 mg/d | CFA | 33.9 ± 0.3^{a} | 32.1 ± 1.8^{b} |
| | Post-CBD | 33.1 ± 0.4^{a} | 31.7 ± 2.1^{b} |
| 62.3 mg/d | CFA | 33.5 ± 0.6^{a} | 34.1 ± 0.8^{a} |
| | Post-CBD | 31.0 ± 1.1^{a} | 30.2 ± 0.9^{a} |

^{a}n = 3;
^{b}n = 5

### 2. Evaluation of the Pain-Related Posture

The abnormal posture of the affected hind limb of each animal was assigned a single score using a subjective pain-related behavioral scale (spontaneous pain rating score 0-5), i.e. 0—normal; 1—curling of the toes, 2—eversion of the paw; 3—partial weight bearing; 4—non-weight bearing and guarding; and 5—avoidance of any contact with the hindlimb (Sluka et al. (1994) Neuroreport 5:109-112).

Pain scores after 6.2 mg/d (1.6±0.5) and 62.3 mg/d (1.7±0.6) doses in CFA post-CBD treated rats were improved (p<0.05) compared to their respective CFA control rats (4.0±0.0 and 3.7±0.6) (Table 3). Pain scores for CFA post-CBD rats 0.6 mg/d (4.0±0.0) and 3.1 mg/d (3.7±0.3) were similar compared to their respective CFA control rats (4.0±0.0 and 3.5±0.0). Normal rats score 0 in this test.

TABLE 3
Assessment of CFA-induced pain rating scores after the application
of CBD or vehicle gel for four consecutive days (day 4)
Spontaneous pain rating scores [mean ± SD]

| | | CFA + PLA | CFA + CBD |
|---|---|---|---|
| 0.6 mg/d | CFA | 4.0 ± 0.0^{a} | 4.0 ± 0.0^{b} |
| | Post-CBD | 4.0 ± 0.0^{a} | 4.0 ± 0.0^{b} |
| 3.1 mg/d | CFA | 4.0 ± 0.0^{a} | 4.0 ± 0.0^{b} |
| | Post-CBD | 3.5 ± 0.0^{a} | 3.7 ± 0.3^{b} |
| 6.2 mg/d | CFA | 4.0 ± 0.0^{a} | 4.0 ± 0.0^{b} |
| | Post-CBD | 4.0 ± 0.0^{a} | 1.6 ± 0.5^{b}* |
| 62.3 mg/d | CFA | 4.0 ± 0.0^{a} | 4.0 ± 0.0^{a} |
| | Post-CBD | 3.7 ± 0.6^{a} | 1.7 ± 0.6^{a}* |

*p < 0.05
^{a}n = 3;
^{b}n = 5

### C. Assessment of Pain Related Behaviors

### 1. Secondary Thermal Hyperalgesia

Secondary thermal hyperalgesia was assessed with the paw withdrawal latency (PWL) test (Hargreaves et al. (1988) Pain 32: 77-88). PWLs were measured in response to a radiant heat source shone on one hind paw of the animals from beneath a glass top table. A shortened PWL response in animals with knee joint inflammation was indicative of secondary thermal hyperalgesia. Animals were placed in separate plastic cubicles (4×4×10 in) on a glass top table (2 mm thickness of glass) and allowed to adapt to their new environment for 20-30 min before testing. A small movable metal box that focused a high-intensity light beam through an aperture (0.5 cm×0.5 cm) was attached to an on/off switch and a digital timer. The light beam was applied to the plantar surface of the hind paw through the glass until the rat lifted its paw. At that time, the light is switched off and the number of seconds on the digital timer recorded as the PWL (sec). The maximal cut off time for the paw-withdrawal reflex was set to 15 sec (55° C.). Thereafter, PWL tests were performed 4 h after CBD application for the consecutive 4 days. Both hind paws were tested independently for 5 trials per side with 5-minute intervals between trials. A mean of the 5 trials was calculated for each animal. The PWL tests were performed before knee joint injection as a baseline and 5 times after the CFA injection beginning on day 3 (days 3-7). The examiner was blind from the treatment group of animals.

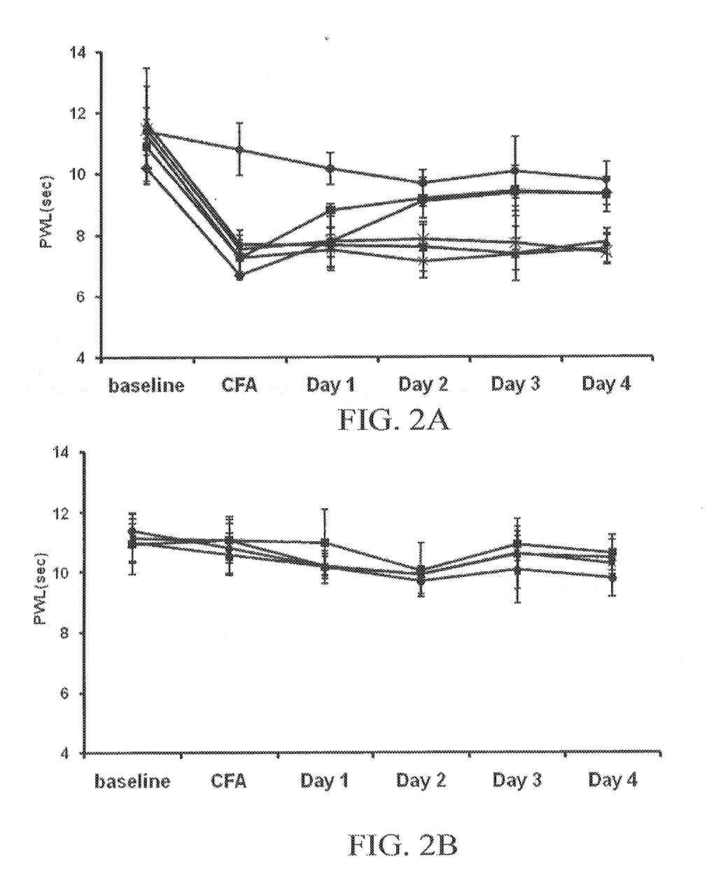

PWLs (sec) performed on the opposite uninflamed (right) paw of mono-arthritic rats showed no differences in baseline across all treatment groups. All CFA rats demonstrated a good response to the CFA, and they had significantly shortened (p<0.05) PWL for the inflamed paw compared to the normal vehicle injected rats. By day 2 of CBD application, a significant improvement (p<0.05) for the 6.2 mg/d (9.2±0.7) and 62.3 mg/d (9.1±0.2) CBD treatments was observed as compared to vehicle gel (7.1±0.6), 0.6 mg/d (7.8±0.5), and 3.1 mg/d (7.6±0.8) treatments (FIG. 2A). There were no significant differences (p>0.05) between the 6.2 mg/d and 62.3 mg/d treatments on day 2. The PWL scores for the 6.2 mg/d and 62.3 mg/d doses were similar to those of the normal vehicle treated rats (9.7±0.4) by the second day of cannabidiol gel application. On day 2, normal rats receiving the vehicle gel (9.7±0.4), or 0.6 mg/d (9.9±0.2), 3.1 mg/d (9.9±0.3), and 6.2 mg/d (10.1±0.9) CBD, all had similar PWL values indicating that the CBD had no effect in normal rats (FIG. 2B). By day 4, CFA rats receiving 0.6 mg/d and 3.1 mg/d CBD still did not have improved PWL scores. The day 4 scores for CFA rats receiving 6.2 mg/d and 62.3 mg/d were equivalent to each other (9.2±0.3 and 9.3±0.2) and to their own baseline scores (10.9±0.3 and 9.3±0.3). In fact, the day 4 values for the normal controls that received vehicle gel (9.4±0.6) and normal controls that received 6.2 mg/d CBD gel (10.6±0.3) were not different.

### 2. Open Field Behavioral Testing

Exploratory behavioral activity of the rats was monitored using the Flexfield Animal Activity System (San Diego Instruments; San Diego, Calif.) with a Photobeam Activity System software coupled to a computer according to previously published methods by Zhang et al., (2004) Pharmacol Biochem Behav 77: 145-153. Briefly, six main parameters were recorded: rearing events and rearing time, active time, and resting time, total activity (i.e. number of photobeams disturbed in the x-y-z planes while the rat was moving around in the box or involved in stationary movement such as grooming), and distance traveled. Activity testing was conducted in an isolated, temperature controlled area at similar times daily in order to provide consistent results.

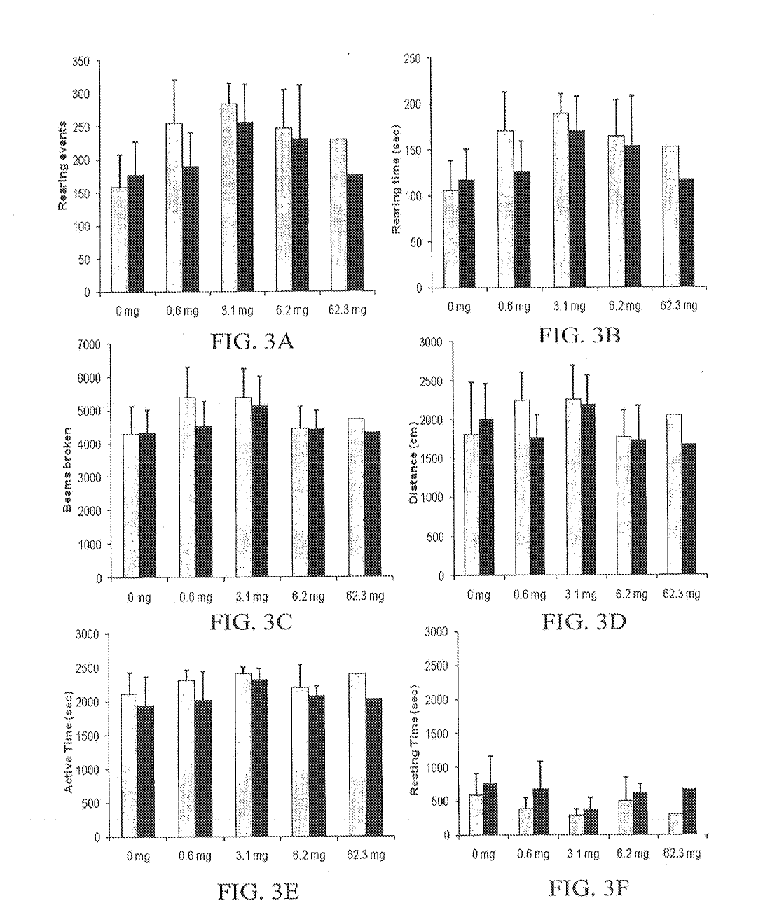

The exploratory activities in normal (non-CFA) rats receiving vehicle gel with or without CBD were assayed and compared. No differences (p>0.05) were determined among the groups for the six parameters except for a difference (p<0.05) between normal rats receiving only vehicle gel (158.3±49.2, 105.4±32.8) and normal rats receiving a 3.1 in& CBD (284.0±31.1, 189.1±20.7) treatment for pre-dose rearing events and rearing time, respectively. No significant (p>0.05) differences were seen when comparing the pre-dose values to the post-dose values among the five treatment groups for the six parameters: rearing events and time, total beam broken, distance traveled, active time, and resting time (FIGS. 3A-3F) except for distance traveled (cm) (p<0.05) with the 0.6 mg/d (pre-dose 2245.5±367.4 compared to post-dose 1757.1±299.2).

### D. Experimental Procedures

### 1. Cannabidiol Administration

Baseline parameters (body weight, knee joint temperature, and knee joint circumference) were obtained before the knee injection of rats with CFA. PWL test and exploratory activity testing were also conducted prior to CFA injection. Rats were anesthetized with a short term anesthetic and the left knee joint, cavity was injected with CFA for induction of mono-arthritis. On day 3, the back of each animal was shaved. On day 4 or 7 following mono-arthritis induction, the following parameters (body weight, knee joint temperature, and knee joint circumference) were again obtained prior to initial skin application of CBD gel or vehicle gel. Rats received daily treatments for four consecutive days of either vehicle gel, or CBD gel. Gel was applied to the skin in a templated region on the dorsal surface of the rat and rubbed into the skin for 30 sec to ensure complete coverage. The nitrile template was used to ensure specific area reproducibility. The PWL test was conducted 4 h after gel application. Exploratory activity was monitored prior to initial gel application and on the third day post gel application (after 6 h). On the final day of gel application after the body and knee joint parameters were obtained and PWL test was conducted, rats were cuthanized by pentobarbital overdose. Blood samples were obtained for CBD plasma quantification, and tissues (knee joints, dorsal root ganglia, spinal cord, brain) were collected after perfusion.

### 2.Experimental Groups

Half of the treated rats were injected with the CFA in the knee joint. Within each group of rats, half received vehicle gel without CBD and the other half received the gel CBD. Four different doses were tested in the rats, 0.6 mg/d, 3.1 mg/d, 6.2 mg/d, or 62.3 mg/d. Doses of CBD containing gel were based on previous studies with a 1% gel in hairless guinea pigs. Rats were initially dosed with CBD at 6.2 mg/d. A two-fold lower dose and a ten-fold lower/higher dose (3.1 mg/d, 0.6 mg/d, and 62.3 mg/d) were also investigated. Area of application, amount of gel, and/or concentration of gel was adjusted to achieve the desired doses. The areas of application and amount of gel applied correspond to the previously mentioned doses: 3.5 cm² (75 μL), 17.5 cm² (375 μL), 35.0 cm² (750 μL of 1% w/w CBD gel), and 35.0 cm² (750 μL of 10% w/w CBD gel).

### 3. Plasma Extraction

50 μL aliquot of plasma collected from cannabidiol dosed rats was added to a siliconized microcentrifuge tube containing 500 μL of 1:1 ACN:ethyl acetate. The plasma/ACN:ethyl acetate sample was vortexed for 30 sec and centrifuged at 10,000×g for 20 min. The supernatant was removed and placed into a clean silanized culture tube and evaporated under nitrogen in a 37° C. water bath. The dried sample was reconstituted with 100 μL of ACN and vortexed for 30 sec, then sonicated for 5 min. The sonicated sample was then placed into an HPLC vial containing a silanized low volume insert and the concentration of CBD in the plasma samples analyzed by LC/MS. Standards were prepared by spiking blank plasma samples with respective standard concentrations and extracting the standards similarly to the collected plasma samples.

The plasma CBD concentrations in rats receiving topical application of each of the four doses, i.e., 0.6 mg/d, 3.1 mg/d, 6.2 mg/d and 62.3 mg/d, were 3.8±1.4 ng/mL (n=9), 17.5±4.4 ng/mL (n=8), 33.3±9.7 ng/mL (n=8), and 1,629.9±379.0 ng/mL (n=4), respectively. Plasma concentrations of CBD from rats dosed with 0.6 mg/d, 3.1 mg/d and 6.2 mg/d displayed an excellent linear pharmacokinetic correlation (slope=l.0, R2=0.999). However, the 62.3 mg/d dose applied did not follow the linear pharmacokinetic profile.

### 4. Analytical LC/MS Method

The LC/MS system used to analyze samples was comprised of a Waters Alliance 2695 pump and autosampler, a Micromass™ ZQ detector, and 996 photodiode array detector with MassLynx™ software (Waters Corp., Milford, Mass.). A Symmetry™ C18 column (150×2.1 mm, 5 μm) with a Sentry Symmetry™ guard column (10×2.1 mm, 3.5 μm) was used with the LC/MS system. The ZQ detector was used with an electrospray ionization (ESI) probe set for single ion monitoring (SIM) for cannabidiol quantification. Analysis was performed in negative mode for m/z 313 [CBD-H]⁻ (dwell time: 30 sec). Capillary and cone voltage were set at 35 kV and 40 V, respectively. Source block and desolvation temperatures were set at 120° C. and 250° C., respectively. Nitrogen produced from the Nitroflow Lab (Parker Hannifin Corp; Cleveland, Ohio) was used as nebulization and drying gas flow rates of 50 and 450 L/h, respectively. Volume injected onto the column was 20 μL and run time was 10 min. The mobile phase was comprised of 75:25 ACN:2 mM ammonium acetate buffer w/5% ACN and used at a flow rate of 0.25 mL/min. Retention time for cannabidiol was 5:6-5.7 min. Standard curves were linear within the range of 2 ng/mL-300 ng/mL.

### 5. Data Analysis

Data are presented as mean±SD in tables and figures. Results were analyzed with Student paired t-test and one way ANOVA followed by Tukey post-hoc analysis to determine if there were significant differences before and after treatment and among treatment groups. SigmaStat™ 2.03 was the statistical program used to calculate results (Systat™ Software, Inc.; Richmond, Calif.). Values of p<0.05 were considered significant.

### E. Discussion

### 1. Transdermal CBD Correlation to Blood Absorption

Plasma concentrations of CBD from rats dosed with 0.6 mg/d, 3.1 mg/d and 6.2 mg/d exhibited an excellent linear correlation; however, the 62.3 mg/d dose did not fit into the linear pharmacokinetic profile. Without wishing to be bound by theory, the failure of the 62.3 mg/d dose to fit into the profile could be due to a change in the absorption (from the formulation change in dose per unit area, drug saturation level in the vehicle, and/or change in partitioning), and/or the achievement of capacity-limited metabolism at this large dose. With the 62.3 mg/d dose, the 10% gel formulation was very close to solubility saturation, which may have caused an increased absorption rate, as compared to the 1% formulations used. In a study by Paudel et al. (unpublished) in hairless guinea pigs dosed with a 1% CBD gel, contact studies were performed to determine the amount of residual drug left on the surface of the skin. Results showed that approximately 15% residual drug remained on the skin surface up to 8 h post application of a hydroalcoholic CBD gel. Higher concentration CBD gels were not examined. Due to the limited surface area available to topically dose a rat, the area for the 6.2 mg/d and 62.3 mg/d doses were the same. Also with 62.3 mg/d, especially given the size of the rat (260-280 g), systemic CBD levels may have been higher than predicted by a linear pharmacokinetic relationship if a zero-order elimination process was reached at this high dose. Paudel et al (unpublished) saw a Cₘₐₓ plasma concentration of 45.8±8.4 ng/mL (n=3) in hairless guinea pigs dosed once with the 6.2 mg/d CBD gel compared to 85.6±48.4 ng/mL (n=5) after 3 consecutive days of gel application. The Tₘₐₓ for a single application to the hairless guinea pigs was 6.7±2.1 h compared to 40.0±14.5 after multiple applications. CBD plasma concentrations were detected out to 120 h after a single initial gel application. The results presented herein indicate that topical administration of CBD to an afflicted area in osteoarthritis is likely to improve pain and inflammation relief by providing a higher tissue concentration of drug at the site of injury.

### 2. Assessment of Knee Joint Inflammation and Nociceptive Responses

Knee Joint Inflammation. A normal knee circumference of a 250 g rat was 5.6 cm. Reduction in the knee joint inflammation has been seen previously with orally administered cannabidiol treatments (Costa et al.(2007) Eur J Pharmacal 556: 75-83). The results presented herein demonstrate that the 6.2 mg/d dose appeared to be the optimal treatment since no further improvement in the knee inflammation was observed with the 62.3 mg/d treatment. A reduction in inflammation is important for the symptomatic treatment of osteoarthritis because osteophyte formation leads to enlargement of the affected joints reducing physical functioning and increasing pain (Buckwalter and Martin, (2006) Adv Drug Deliv Rev 58: 150-167; Jones et al. (2000) J Rheumatol 27: 745-752. No knee joint temperature differences were measured among treatment groups. Barak et al. (1992) Biotherapy 4: 317-323 and Lu et al. (2008) Eur J Neurosci 27:1153-1165 had similar results for other agents that reduced inflammation with no temperature differences measured between control groups and treated groups of rats using the CFA arthritis model.

Spontaneous Pain Rating Scores. Pain caused by osteoarthritis is typically the reason individuals first seek medical treatment. A successful treatment for osteoarthritis should not only reduce inflammation but also eliminate pain. Spontaneous pain related posture scores (scale 1-5) improved (p<0.05) for CFA rats treated with CBD 6.2 mg/d (pre-dose 4.0±0.0 to post-dose 1.6±0.6) and 62.3 mg/d (pre-dose 4.0±0.0 to post-dose 1.7±0.6) compared to their respective CFA controls receiving vehicle gel (pre-dose 4.0±0.0 to post-dose 4.0±0.0 and pre-dose 4.0±0.0 to post-dose 3.7±0.6). The 6.2 mg/d and 62.3 mg/d treatments were similar in their reduction of pain scores.

Secondary Thermal Hyperalgesia. An improvement in PWL was seen in the 6.2 mg/d and 62.3 mg/d doses. However, the improvement in PWL was similar between the 6.2 mg/d dose and 62.3 mg/d. Malfait et al. (2000) (Proc Natl Acad Sci USA 97: 9561-9566) reported similar results in mice with collagen-induced arthritis, a rheumatoid arthritis model, treated with cannabidiol either i.p. or orally. The highest treatment dose of CBD administered also did not perform as well as a lower treatment in their studies. Of the i.p. treatments they administered (2.5 mg/kg, 5 mg/kg, 10 mg/kg, or 20 mg/kg), 5 mg/kg was optimal for arthritis suppression in mice and for orally administered treatments (10 mg/kg, 25 mg/kg or 50 mg/kg), 25 mg/kg was optimal (Malfait et al. (2000)). Costa at al. (2004a) Naunyn-Schmiedebergs Arch Pharmacol 369: 294-299 saw vast improvements in PWL in mice with 10 mg/kg, 20 mg/kg, and 40 mg/kg oral CBD administration at 3 h post carrageenan-induced paw edema with the elimination of hyperalgesia. Even by 6 h, the lower two doses administered orally, 5 mg/kg and 7.5 mg/kg, had eliminated hyperalgesia. For comparison, the transdermal doses used for the current studies would have been approximately 2.3 mg/kg, 11.5 mg/kg, 23.0 mg/kg, and a 230.0 mg/kg dose. The results presented herein demonstrate an improvement (p<0.05) in PWL for rats with CFA induced mono-arthritis at 4 h (d 1) post CBD dosing with the 6.2 mg/d dose compared to the CFA rats receiving vehicle gel. The improvement continued for the duration of the study. By day 2, no differences (p>0.05) in PWL were seen between the normal rats and rats receiving 6.2 mg/d and 62.3 mg/d transdermal CBD.

### 3. Assessment of Psychoactive Effects

Exploratory Behavioral Activity. During exploratory behavioral testing, no differences in activity measurements were determined in normal rats between pre-CBD and after four days of CBD administration except for distance traveled (cm) (p<0.05) with the 0.6 mg/d (pre-dose 2245.5±367.4 compared to post-dose 1757.1±299.2). CBD is a known non-psychoactive drug, and due to its low affinity for the CB1 receptor (Croxford (2003) CNS Drugs 17: 179-202) it would be expected that exploratory behavioral activity would remain similar among treatment groups, including control rats. Costa et al. (2003) First Eur Workshop on Cannabinoid Research. Madrid (Spain) demonstrated that a CB1 receptor antagonist had no effect on the anti-inflammatory and anti-hyperalgesic effects of CBD. Behavioral changes with CBD treatments were not expected due to the safe nature of the drug compared to the negative side effects associated with THC. THC has been shown to cause hypothermia and hypomobility (Zimmer et al. (1999) Proc Nall Acad Sci USA 96: 5780-5785) which are avoided with the use of CBD. Therefore with CBD treatments, changes particularly in active and resting times would have been observed if CBD's pharmacological actions were similar to THC.

These disclosure presented herein indicate that transdermal CBD has long lasting effect and alleviates the symptoms of arthritis, particularly osteoarthritis.

We claim:

1. A method for relieving symptoms associated with traumatic injury or disease conditions in a subject in need thereof comprising the steps of:
(a) selecting at least one cannabinoid from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2 or prodrug thereof;
(b) selecting a permeation enhancer from the group consisting of isopropyl myristatc, propylene glycol monolauratc, diethylene glycol monoethyl ether, an oleoyl macrogolglyceride, a caprylocaproyl macrogolglyceride, and an oleyl alcohol, ethanol and oleic acid,
(b) combining an amount of the selected cannabinoid with an amount of the permeation enhancer and an amount of a pharmaceutically acceptable gel forming material and initiating formation of a cannabinoid-containing gel, and
(c) applying an amount of the cannabinoid-containing gel transdennally to a subject in need thereof,
wherein the cannabinoid-containing gel contains is applied to skin of the subject for a sufficient time to alleviate inflammation and pain associated with the traumatic injury or disease condition.

2. The method of claim 1 wherein the traumatic injury is a sprain, a strain or a contusion and the disease condition is osteoarthritis.

3. The method of claim 1, wherein the cannabinoid is a combination of cannabinoids selected from the group consisting of: cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

4. The method of claim 1; wherein the cannabinoid is cannabidiol.

5. The method of claim 1, wherein the pharmaceutically acceptable gel forming material is selected from the group consisting of anionic polymers.

6. The method of claim 5 wherein the anionic polymer is a polyacrylic acid, carboxypolymethylene, or carboxymethylcellulose.

7. The method of claim 6, where in the anionic polymer is selected from the group consisting of CARBOPOL™, a derivative of CARBOPOL™ polymers, PEMULEN™, NOVEON™ and polycarbophils.

8. The method of claim 7 wherein the derivative of Carbopol is selected from the group consisting of Carbopol™ Ultrez 10, Carbopol™, Carbopol™ 941, Carbopol™ 954, Carbopol™ 980, Carbopol™ 981, Carbopol™ ETD 2001, Carbopol™ EZ-2 and Carbopol™ EZ-3.

9. The method of claim 1 wherein the permeation enhancer is ethanol.

10. The method of claim 1, wherein the cannabinoid is cannabidiol and the permeation enhancer is selected from the group consisting of ethanol and isopropyl myristate.

11. The method of claim 1, wherein the cannabinoid-containing gel is delivered to the subject's skin to an area not affected by osteoarthritis.

12. The method of claim 11, wherein the area not affected by osteoarthritis is the subject's back, abdomen, chest or upper arms.

13. The method of claim 1, further comprising the steps of selecting an opiate; and delivering the selected opiate transdermally with the selected cannabinoid.

14. The method of claim 1 wherein the symptom of osteoarthritis is at least one of inflammation or pain.

15. The method of claim 1 wherein the cannabinoid is rubbed into the skin for sufficient time for the cannabinoid-containing gel to be almost completely absorbed into the skin.

16. The method of claim 1 wherein the amount of cannabinoid delivered to the skin of the subject is sufficient to produce systemic levels of the cannabinoid of about 36mg/d.

17. The method of claim 1, wherein the permeation inhibitor is ethanol and step (c) further comprises
(i) combining the cannabinoid, gel forming material and ethanol with water and isopropyl myristate to form a solution, then
(ii) adding a sodium hydroxide solution to the solution of (i).

18. The method of claim 17 wherein the amount of cannabinoid is about 1% w/w, the amount of gel forming material is about 0.9% w/w, the amount of ethanol is about 72%, the amount of water is about 20%, the amount of isopropyl myristate is about 0.5% w/w, and the amount of sodium hydroxide is about 0.5%, wherein the sodium hydroxide solution is a 0.1 N sodium hydroxide solution.

19. A cannabinoid-containing gel formulation comprising about 1% w/w-10% w/w cannabinoid, about 1% w/w of a pharmaceutically acceptable gel forming material, about 73% w/w of a vehicle or permeation enhancer, about 20% w/w water, and about 0.5% sodium hydroxide, wherein the sodium hydroxide solution is a 0.1 N sodium hydroxide solution.

20. The cannabinoid-containing gel formulation of claim 19 wherein the cannabinoid is cannabidiol, the gel forming material is Carbopol™ 980, and the permeation enhancer is ethanol and isopropyl myristate
